(19) United States
(12) Patent Application Publication
(10) Pub. No.: US 2011/0131839 A1
(43) Pub. Date: Jun. 9, 2011

(54) Pneumatic Alternating Pressure Relief of a Foot

(75) Inventors: Benny Ballin, Ramat-Gan (IL); Noam Calderon, Haifa (IL); Alexander Salman, Haifa (IL)

(73) Assignee: C-Boot Ltd.

(21) Appl. No.: 12/957,681

(22) Filed: Dec. 1, 2010

Related U.S. Application Data
(60) Provisional application No. 61/266,317, filed on Dec. 3, 2009.

(51) Int. Cl. A61F 5/34

(52) U.S. Cl. 36/141

(57) ABSTRACT

A system for alternately applying and relieving pressure of predetermined portions of a foot of a user. The system includes a plurality of inflatable chambers, located at predetermined locations, corresponding to the predetermined portions of the foot and a pump. The system also includes a center pneumatic valve, coupled with the pump and with the inflatable chambers, for receiving air from the pump and inflating at least one of the inflatable chambers. The system further includes a pneumatic valve assembly, coupled with the center pneumatic valve. The pneumatic valve assembly is coupled with at least a respective one of the inflatable chambers. The pneumatic valve assembly is moveable between two positions, and changes position when a predetermined pressure value is achieved in the corresponding inflatable chamber. When the pneumatic valve assembly changes position, the center pneumatic valve changes position, thereby inflating another inflatable chamber.

### REFERENCE TO RELATED APPLICATIONS

This application claims the benefit of and priority to co-pending U.S. Patent Application Ser. No. 61/266,317, filed Dec. 3, 2009, the entire disclosure of which is incorporated by reference herein.

### FIELD OF THE INVENTION

The disclosed technique relates to pressure relief of a foot of a user, in general, and to methods and systems for relieving pressure of selected portions of a user's foot during walking, using alternating pneumatic pressure to alternately inflate and deflate predetermined chambers, in particular.

### BACKGROUND

The action of treading, walking, running and the like causes pressure appliance on certain portions of the walker's foot. The location of these portions is substantially constant, depending on the shape of the walker's foot, the shoe and the sole, and the type of activity performed (i.e., walking, treading, running, jumping and the like). Shoes and shoe soles for pressure relief of certain portions of the foot are known in the art. Diabetic patients are often subject to the appearance of Diabetic Foot Ulcers (DFU), caused due to poor vascular blood flow combined with delayed wound healing. In extreme cases, such DFUs may present a risk of limb amputation for the patient, in case of infection and delayed healing. Shoe soles for relief and treatment of DFUs is known in the art.

U.S. Pat. No. 7,331,121, issued to Lo and entitled “Inflatable Shoe Sole” is directed to a shoe sole including an inflatable chamber. The chamber is inflated by a pump via a connection tube. The connection tube includes a first valve to prevent air from flowing back into the pump and an inlet tube extends from the pump to introduce air from outside into the pump. A distal end of the connection tube includes a plurality of outlets, located corresponding to toes of the wearer. The connection tube has a neck portion which defines a narrowed path in the connection tube so that air is stopped at the neck portion to inflate the chamber. When the pressure of the air reaches a certain pre-decided value, air passes through the neck portions and blows out from the outlets to cool the toes of the wearers. The neck portion in the tube allows the pump to pump air into the chamber under certain pressure. The air to be released from the tube is released when the pressure is higher than that in the pumping device.

U.S. Pat. No. 6,430,843 issued to Potter et al. and entitled “Dynamically-controlled cushioning system for an article of footwear” is directed to dynamically distributing and regulating pressure within a fluid-filled bladder in a shoe. The cushioning system includes a sealed, fluid-filled bladder formed with a plurality of separate cushioning chambers, and a control system. The control system includes a CPU, pressure sensors and valves, and controls fluid communication between the chambers to dynamically adjust the pressure in the cushioning chambers for various conditions such as the activity that the footwear is used in, the weight of the individual and the individual's running style. Fluids in the bladder may include hexafluorethane, sulfur hexafluoroide, nitrogen, air, or other gases.

The control system is in communication with the modulating valves to vary the opening of each of the valves and thus the level of fluid communication of each chamber with the other chambers. As the modulating valves are preferably solenoids (and thus electrically controlled), the control system is in electrical communication with modulating valves. When the CPU controls the pressure in the reservoirs and the tubes, it adjusts the degree of pressurization and thus the stiffness of each support chamber, to provide customized cushioning at different locations of the shoe, without adding or leaking gas from the bladder. Gas may be transferred from any one of the chambers to any of the other chambers to increase or decrease the stiffness of the bladder at a desired location, to thereby tune the overall cushioning characteristics of the midsole for a particular activity or for a specific gait characteristic of the wearer.

U.S. Pat. No. 5,353,525 issued to Grim and entitled “Variable support shoe” is directed to a support for an athletic shoe including air bladders and a pump. The athletic shoe includes a substantially flat pump chamber in the sole of the shoe under the heel of the user, with a one-way valve permitting the drawing in of air when pressure is taken of the heel. The shoe also includes a second one-way valve at the outlet from the flat pump which acts as air is being exhausted from the chamber. One or more pressure bladders for receiving air from the pump are mounted in the sidewalls of the shoe toward the rear thereof adjacent the ankle. When the user is active (i.e., walking or running), the pump is automatically actuated to inflate the air bladders and to provide additional support for the foot and the ankle. The air bladders may be provided with a relief valve to prevent overpressure, and/or with arrangements for slowly leaking air out of the bladder so that when the user is resting, pressure on the foot and ankle is minimized.

U.S. Pat. No. 6,014,823 issued to Lakic and entitled “Inflatable sole lining for shoes and boots” is directed to an inflatable lining for selected inner surfaces of footwear. The inflatable lining is formed of first and second plastic sheets having the shape and size of the sole, or a portion of the upper surfaces of the footwear. The sheets are bonded together in a continuous seam about their peripheral edges thereby forming a sealed interior chamber. A plurality of discontinuous seams are formed between the first and second sheets to create within the sealed interior chamber a plurality of interconnecting tubular passageways. One of the continuous seams can be provided to subdivide the interior chamber into two or more subdivided interior chambers.

The inflatable liner is provided with an air pump with an inlet valve which discharges into a flexible tube which extends to a pressure control valve and then to the interior chamber of the inflatable inner sole. The pump is mounted at the heel of the inner sole. The liner also includes a plurality of flexible tubes, one each connecting the outlet port of its respective valve to a selected one of said sealed chambers. The liner is self-inflated by normal walking or other activities of the wearer, pressing on the pump on the heel portion of the liner. The pressure relief valve is manually adjustable to control the pressure within the inflatable inner sole. Excess air from the pressure control valve can be directed into channels formed on the undersurface of the inner sole where it discharges through sealed apertures in the inner sole, thereby providing forced air circulation in the footwear.

The relief valves automatically relieve pressure in their respective chambers, and the valves are manually adjustable to permit variation of the relief pressures, thus providing a controlled adjustability of the pressures within each chamber of the inflatable liner. The connection of the flexible tubes can be altered to provide variation in the pressure which is applied to any of the chambers, thereby adapting the footwear to different applications, or varying the comfort of various parts of the footwear.

U.S. Pat. No. 7,395,614 issued to Bailey et al. and entitled “Intelligent footwear” is directed to a controllable footwear structure. The structure controls splitting of a force exerted on the sole of the footwear between a first portion, which is stored in an energy storage structure and later returned to the sole, and a second portion which is dissipated. The structure includes a control for controlling the structure in dependence on an activity of the wearer, to alter a relation between the first portion and the second portion, to thereby alter a dynamic characteristic of the footwear. The system provides two distinct systems for adjusting the fit of the shoe. First, a hydraulic system is used to fill bladders for contour and piston actuators for tensioning. Second, a pneumatic system is used to fill bladders and reactive energy chambers within the sole for control over dynamic properties and pressure around the foot. With respect to the pneumatic compressor, a pancake shaped bladder is formed near the heel of the shoe. As weight is applied to the heel, the bladder pressurizes. A set of check valves controls flow direction. Rebound of the pump bladder is by way of a proximate gas pressurized toroidal ring.

The sole of the shoe, below the pressure sensing pad, includes a set of hydraulic bladders. For example, four anatomical zones are defined, each having a bladder space. A set of pneumatic structures is also provided within the sole; however, these are preferably static, as is conventional. If desired, one or two pneumatic structures within the sole may be dynamically controlled during use, for example to balance energy recovery and stability. Each step of the wearer allows a different zone of the shoe to be adjusted. Since the hydraulic and pneumatic systems are separate, each position of the rotary valve allows separate actuation of a respective hydraulic and pneumatic zone.

U.S. Pat. No. 7,258,676 issued to Calderon et al. and entitled “Device and Method for Low Pressure Compression and Valve for Use in the System” is directed to self-powered compression devices and methods for promoting circulation by applying circulative low pressure compression. The system includes a ladder-like support structure for a plurality of sleeves or balloons, which are positioned and tuned such that the first sleeve has the highest pressure and each one above it has a lower pressure than the one below. This is performed by adjusting the magnetic field of control valves adapted for controlling low-pressure compression. The sleeves are placed along the limb such that the pressure is maintained the highest in the first sleeve, the pressure in the second sleeve is equal to or lower than the pressure in the first sleeve, and the pressure in the third sleeve is equal to or lower than the pressure in the second sleeve.

U.S. Pat. No. 6,589,194 issued to Calderon et al. and entitled “Self-powered Compression Devices and Methods for Promoting Circulation and Therapeutic Compression” is directed to a self-powered compression device which includes a plurality of inflatable pockets that form a sleeve around the limb to apply a controlled level of circular compression to the limb. The inflatable pneumatic sleeves wrap around the limb adopting the wearer's shape of the limb. Energy is generated by the wearer's muscle action, weight bearing gravity force and constant transfer of body weight during walking, moving or flexing of the limbs. The energy generated is transmitted to the air or liquid inflated sleeves which surround the limb. As a result of the cyclical and sequential movement of the pockets from the lower portion of the limb towards its upper portion, vector forces are generated. The resulting compression preserves the venous reflex and aids venous flow back from the foot in the direction of the heart. The cyclical pumping action causes the pocket furthest from the heart to inflate first and reach a preset pressure, followed by inflation of the successive sleeves.

U.S. Patent Application Publication No. 2005/0027221 to Calderon et al. and entitled “Device and Method for Low Pressure Compression and Valve for Use in the System” is directed to a self-powered compression device which includes a plurality of inflatable sleeves, a foot pump and a device for distributing compressed air from a compressed air source to the sleeves. The device further includes a ladder-like support structure having a plurality of valve bodies, a plurality of inlet valves for connecting the valve bodies to the compressed air source, a plurality of outlet valves, each adapted to communicate with at least one sleeve that uses compressed air, and a plurality of exhaust valves outside the device. The ladder-like support structure includes a means for providing decreasing pressure in each of the inter-connected sleeves, positioned above the sleeve near the foot, such that the highest pressure is in the first sleeve and the lowest pressure is in the last sleeve.

The pneumatic control system of the device generates a cycle of air flow by closing the exhaust valve in each sleeve, allows air to fill the sleeve through the inlet valve to a pretuned pressure, and finally deflates the sleeve by letting the air flow through the outlet valve. This creates a pressure gradient that facilitates the massaging movement on the limb towards direction of the heart. The magnetically adjustable valves are moveable in each valve body, and adapted for generating low pressure that can be used to create a massaging action without the disadvantage of causing extreme constriction of the swollen body part.

### SUMMARY OF THE INVENTION

It is an object of the disclosed technique to provide novel system for alternately applying and relieving pressure of predetermined portions of a foot of a user, and devices for use in such systems. In accordance with the disclosed technique, there is thus provided a system for alternately applying and relieving pressure of predetermined portions of a foot of a user. The system includes a plurality of inflatable chambers, located at predetermined locations, corresponding to the predetermined portions of the foot and a pump. The system also includes a center pneumatic valve, coupled with the pump and with the inflatable chambers, for receiving air from the pump and inflating at least one of the inflatable chambers. The system further includes a pneumatic valve assembly, coupled with the center pneumatic valve. The pneumatic valve assembly is coupled with at least a respective one of the inflatable chambers. The pneumatic valve assembly is moveable between two positions, and changes position when a predetermined pressure value is achieved in the corresponding inflatable chamber. When the pneumatic valve assembly changes position, the center pneumatic valve changes position, thereby inflating another inflatable chamber.

In accordance with another embodiment of the disclosed technique, there is provided a system for alternately applying and relieving pressure of predetermined portions of a foot of a user. The system including a plurality of inflatable chambers, located at predetermined locations, corresponding to the predetermined portions of the foot. The system also includes a pneumatic valve assembly, coupled with at least a respective one of the inflatable chambers. The pneumatic valve assembly includes at least an entry port and electric command ports for each one of the inflatable chambers. The pneumatic valve assembly is movable between an inflating position and a deflating position for each one of the inflatable chambers. The system further includes a plurality of pressure sensors, each coupled with a respective inflatable chamber, for providing a pressure value of the pressure in the respective inflatable chamber. The system also includes a pump, coupled with an entry port of each of the pneumatic valve assembly, for providing air into the valve assembly, and inflating at least one of the inflatable chambers. The system further includes a processor, coupled with each of the pressure sensors, and with each of the electric command ports. The controller determines if the pneumatic valve assembly is to be in an inflating position or in a deflating position for each one of the inflatable chambers, according to the respective pressure value of each of the inflatable chambers. The processor automatically moves the pneumatic valve assembly into the desired position by providing the necessary electric power to the command ports.

In accordance with another embodiment of the disclosed technique, there is provided a device for use in a system for alternately applying and relieving pressure of predetermined portions of a foot of a user. The device is moveable between a plurality of positions. The system includes a plurality of inflatable chambers, located at predetermined locations, corresponding to the predetermined portions of the foot. The device includes a plurality of membranes, an elongated rod, having a first end and a second end, the rod being coupled with the a first membrane proximate to the first end thereof. The rod is coupled with a second membrane proximate to the second end thereof. The rod being moveable between a plurality of positions. The device also includes a plurality of fasteners, each fastener maintaining the rod in another one of the plurality of positions.

The device further includes a device inlet, coupled with a pump for pumping air into the device inlet, and a plurality of device outlets. Each of the device outlets is coupled with a respective inflatable chamber, for guiding pumped air into the respective inflatable chamber, when the device outlet is open. The device also includes a plurality of exhaust inlets, each coupled with a respective inflatable chamber, for exhausting air from the respective inflatable chamber out of the device, when the chamber inlet is open. The device moves between the plurality of positions, according to the air pressure in the inflatable chambers. Each of the membranes allows or prevents air passage to the inflatable chambers, according to the position the device is at.

In accordance with another embodiment of the disclosed technique, there is provided a device for use in a system for alternately applying and relieving pressure of predetermined portions of a foot of a user. The device is moveable between a plurality of positions. The system includes a plurality of inflatable chambers, located at predetermined locations, corresponding to the predetermined portions of the foot. The system further includes a pneumatic valve, having a plurality of outlets, each coupled with a respective inflatable chamber, for guiding air into a respective inflatable chamber, when the outlet is open. The device includes a ratchet mechanism, for moving the pneumatic valve from one inflatable chamber to another, and an air piston. The air piston is coupled with a pump for pumping air into the device. The air piston includes an air outlet for providing air into an inlet of the pneumatic valve. The device also includes an elongated rod, having a first end and a second end, the first end of the rod is coupled with the air piston. The second end of the rod interacts with the ratchet mechanism, thereby rotating the ratchet mechanism by predetermined increments, when sufficient pressure is applied on the rod by the air piston. The device moves between the positions, according to the ratchet mechanism.

In accordance with another embodiment of the disclosed technique, there is provided a device for use in a system for alternately applying and relieving pressure of predetermined portions of a foot of a user. The device is moveable between two positions. The system includes a plurality of inflatable chambers, located at predetermined locations, corresponding to the predetermined portions of the foot. The device includes an air piston, coupled with an air inlet of the device. The air piston includes a guiding tooth and a piston air outlet, for letting air out of the piston. The device also includes an elongated rod, firmly coupled with the air piston, and a ratchet mechanism, slidably coupled with the air piston and the elongated rod. The ratchet mechanism interacts with the guiding tooth of the piston at each of the positions of the device.

The device further includes a first chamber inflating outlet, coupled with an inlet of a first plurality of the inflatable chambers, for moving air from the piston toward the first inflatable chambers, when the device is at the first position. The device also includes a second chamber inflating outlet, coupled with an inlet of a second plurality of the inflatable chambers, for moving air from the piston toward the second inflatable chambers, when the device is at the second position. The device also includes a first chamber deflating outlet, coupled with an outlet of the first plurality of the inflatable chambers, for deflating air from the first inflatable chambers, when the device is at the second position. The device further includes a second chamber deflating outlet, coupled with an outlet of the second plurality of the inflatable chambers, for deflating air from the second inflatable chambers, when the device is at the first position. The guiding tooth interacts with the ratchet mechanism, thereby rotating the ratchet mechanism by predetermined increments. The device moves between the positions, according to the position of the piston relative to the chamber inflating outlets.

### BRIEF DESCRIPTION OF THE DRAWINGS

The disclosed technique will be understood and appreciated more fully from the following detailed description taken in conjunction with the drawings in which:

FIG. 1A is a schematic illustration of a system, constructed and operative in accordance with an embodiment of the disclosed technique;

FIG. 1B is a side view schematic illustration of the system of FIG. 1A;

FIG. 1C is a perspective view schematic illustration of the system of FIGS. 1A and 1B, set within a shoe;

FIG. 2A is a schematic illustration of a system, constructed and operative in accordance with another embodiment of the disclosed technique;

FIG. 2B is a side view schematic illustration of the system of FIG. 2A;

FIG. 3 is a schematic illustration of a system, constructed and operative in accordance with a further embodiment of the disclosed technique;

FIG. 4A is a schematic illustration side view of a pneumatic control device, in a first position, constructed and operative in accordance with another embodiment of the disclosed technique;

FIG. 4B is a schematic illustration of the pneumatic control device of FIG. 4A, in a second position;

FIG. 5A is a schematic illustration of a system, in a first position, constructed and operative in accordance with a further embodiment of the disclosed technique;

FIG. 5B is a schematic illustration of the system of FIG. 5A, in a second position;

FIG. 6A is a perspective view schematic illustration of a device, in a first position, constructed and operative in accordance with another embodiment of the disclosed technique;

FIG. 6B is a side view schematic illustration of the device of FIG. 6A, in a second position;

FIG. 7 is a schematic illustration of a system, constructed and operative in accordance with a further embodiment of the disclosed technique; and

FIG. 8 is a schematic illustration of a system, constructed and operative in accordance with another embodiment of the disclosed technique.

### DETAILED DESCRIPTION

The disclosed technique overcomes the disadvantages of the prior art by providing a system and method for pressure relief portions of a foot of a user, by alternately applying and relieving pressure of selected portions of a user's foot during walking, using alternating pneumatic pressure. The system includes a plurality of inflatable chambers, possibly arranged in groups of chambers. Each chamber or group of chambers is inflated and deflated alternately, for example, by a respective pneumatic valve. Air is supplied to inflate the chambers by a manual pump coupled with an elastic flexible element (e.g., a spring or other specific self retained geometry) located below the heel portion of the foot. The walking action of the user operates on the flexible element to activate the pump (e.g., pressing the flexible element), thereby providing a self-powered compression system.

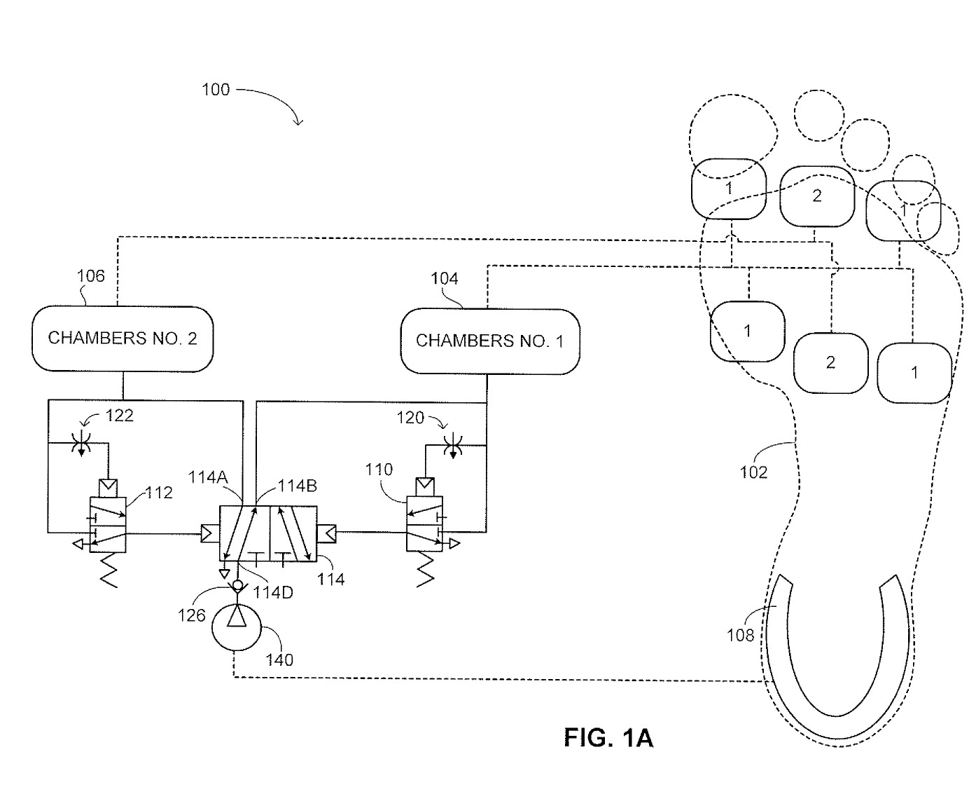

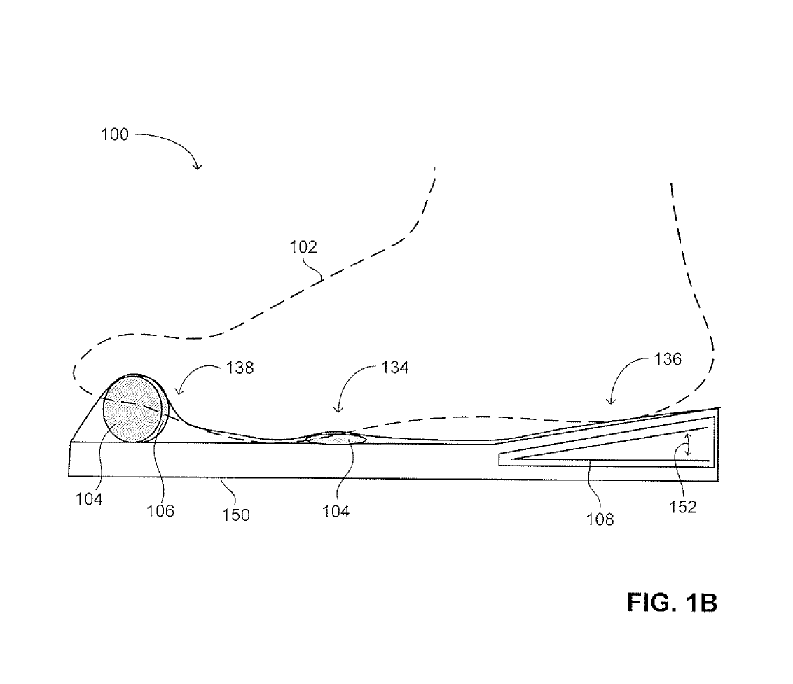

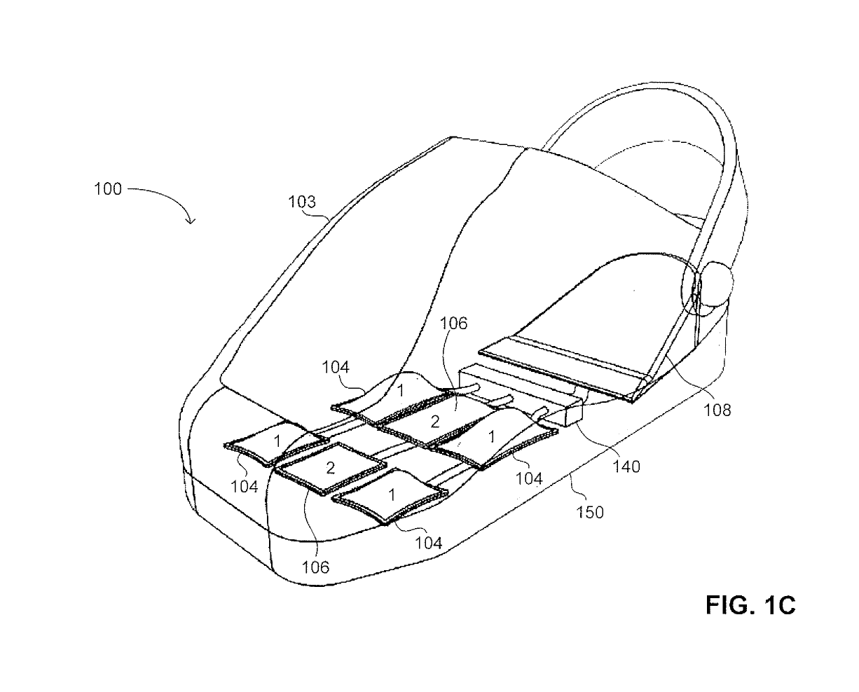

Reference is now made to FIGS. 1A, 1B and 1C. FIG. 1A is a schematic illustration of a system, generally referenced 100, constructed and operative in accordance with an embodiment of the disclosed technique. FIG. 1B is a side view schematic illustration of the system of FIG. 1A. FIG. 1C is a perspective view schematic illustration of the system of FIGS. 1A and 1B, set within a shoe. System 100 is a system for alternately applying and relieving pressure of predetermined portions of a user's foot 102. System 100 includes a first group 104 of inflatable chambers (marked “No. 1”), a second group 106 of inflatable chambers (marked “No. 2”), a spring 108, an air pump 140, a first pneumatic valve 110, a second pneumatic valve 112, a center pneumatic valve 114, a first regulator 120, a second regulator 122, a check valve 126, and a housing 150 (FIG. 1B). Spring 108 is coupled with pump 140. Center pneumatic valve 114 is coupled with pump 140 via check valve 126, first pneumatic valve 110, second pneumatic valve 112, first group 104 of inflatable chambers, and with second group 106 of inflatable chambers. First pneumatic valve 110 is further coupled with first group 104 of inflatable chambers, via first regulator 120. Second pneumatic valve 112 is further coupled with second group 106 of inflatable chambers, via second regulator 122. The components of system 100, except for the inflatable chambers, are located within housing 150. Housing 150 may be located, for example, within a sole of a shoe, such as a shoe 103 of FIG. 1C, worn by the user. First pneumatic valve 110 and Second pneumatic valve 112 may be considered to be part of a pneumatic valve assembly (not shown). It is noted that the pneumatic valve assembly may include more than two pneumatic valves, according to the properties and characteristics of the system.

With reference to FIG. 1B, spring 108 is located below a heel region 136 of a foot 102 of a user. Spring 108 is depicted as a folding spring, moving in a direction indicated by a double arrow 152, according to pressure applied thereon by the heel of foot 102. Alternatively, spring 108 may be replaced with any other known elastic flexible element, which would operate pump 140, when pressure is applied thereon. The chambers of first group 104 of inflatable chambers and of second group 106 of inflatable chambers are located below a front region 138 and a center region 134 of foot 102. Each of first pneumatic valve 110 and second pneumatic valve 112 is depicted as a 3/2 pneumatic valves. Center pneumatic valve 114 is depicted as a 5/2 pneumatic valve, wherein two positions are available, a first position (shown in FIG. 1A), and a second position (not shown).

When the heel of foot 102 applies pressure on spring 108 (i.e., when the user treads, walks or runs), spring 108 compresses air into pump 140. Pump 140 pumps the compressed air and forwards the air into a port 114D of center pneumatic valve 114, through check valve 126. Check valve 126 prevents air from flowing back toward pump 140. Center pneumatic valve 114 forwards the compressed air through port 114B into first group 104 of inflatable chambers, thereby inflating these chambers. Meanwhile, chambers No. 2 are deflated of the air therein through port 114A of center pneumatic valve 114.

The compressed air is also supplied to regulator 120. As long as valve 110 does not change its position (and therefore valve 114 does not change its position), the compressed air further inflates first group 104 of inflatable chambers. When the pressure activates first pneumatic valve 110, it changes its position, thereby changing the position of center pneumatic valve 114 from the first position to the second position.

In the second position, center pneumatic valve 114 forwards the compressed air from pump 140 through port 114A into second group 106 of inflatable chambers, thereby starting to inflate these chambers. Meanwhile, chambers No. 1 are deflated of the air therein through port 114B of pneumatic valve 114. In this position, the compressed air is also supplied to regulator 122. As long as valve 112 does not change its position (and therefore valve 114 does not change its position), the compressed air further inflates second group 106 of inflatable chambers. When the pressure activates second pneumatic valve 112, it changes its position, thereby changing the position of center pneumatic valve 114 from the second position back to the first position. Then, the chambers of second group 106 of inflatable chambers are deflated, while the chambers of first group 104 of inflatable chambers are inflated again. In this manner, the groups of chambers are alternately inflated and deflated, according to the air pressure created therein. Whenever a group of chambers is inflated or deflated, pressure is applied to- and relieved off- of the corresponding parts of the user's foot. For example, such controlled pressure application and relief is useful for treating or relieving pain induces by diabetic foot ulcers of the user. Alternatively, such controlled pressure application and relief may be employed by a healthy user to promote blood flow in the foot or simply providing an enjoyable feeling to the user.

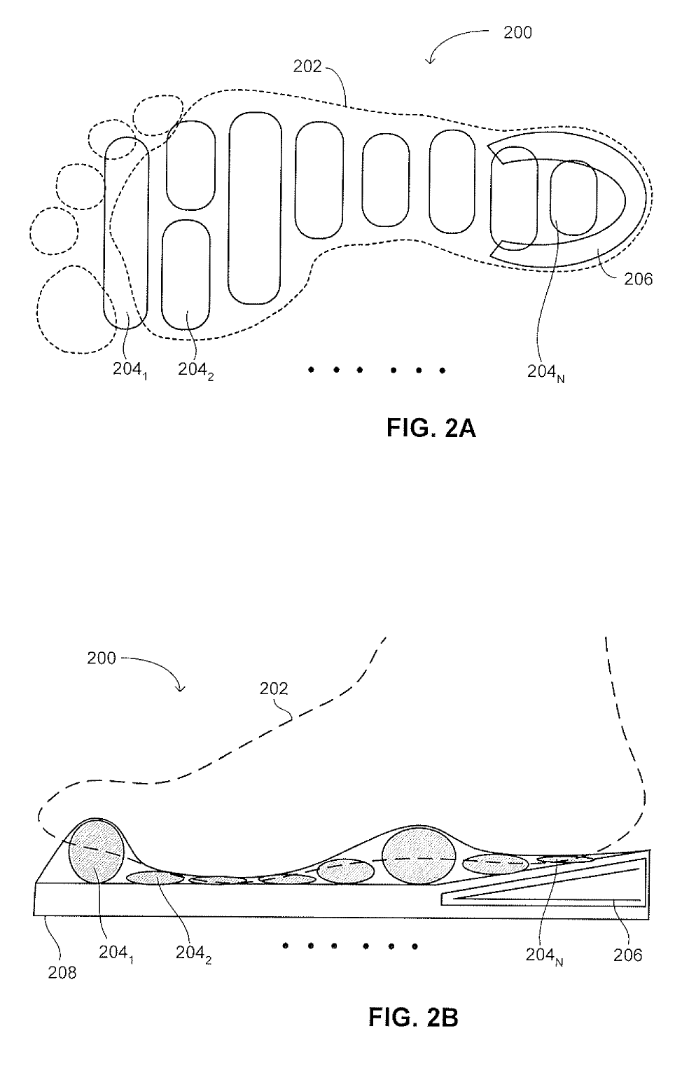

Reference is now made to FIGS. 2A and 2B. FIG. 2A is a schematic illustration of a system, generally referenced 200, constructed and operative in accordance with another embodiment of the disclosed technique. FIG. 2B is a side view schematic illustration of the system of FIG. 2A. System 200 includes a plurality of groups 204₁, 204₂ . . . , 204_{N} of inflatable chambers, arranged along a foot 202 of a user. System 200 is similar to system 100 of FIGS. 1A and 1B. System also includes a spring 206 (or any other type of elastic flexible element, for example with a self retained geometry) for providing air into a pump (not shown) of system 200. Spring 206 is located at the heel portion of foot 202, similarly to system 100.

Similarly to the above description of system 100, the pump provides air through a pneumatic valve (not shown) into a first group of inflatable chambers 204₁, until a predetermined pressure is achieved in these chambers. Then, the pneumatic valve changes its position, thereby providing the air into another group of inflatable chambers, for example, 204₂, and so on. Thus, different inflatable chambers are alternately inflated and deflated, according to a predetermined pressure scheme, thereby applying pressure to—and relieving pressure off—of the corresponding parts of the user's foot.

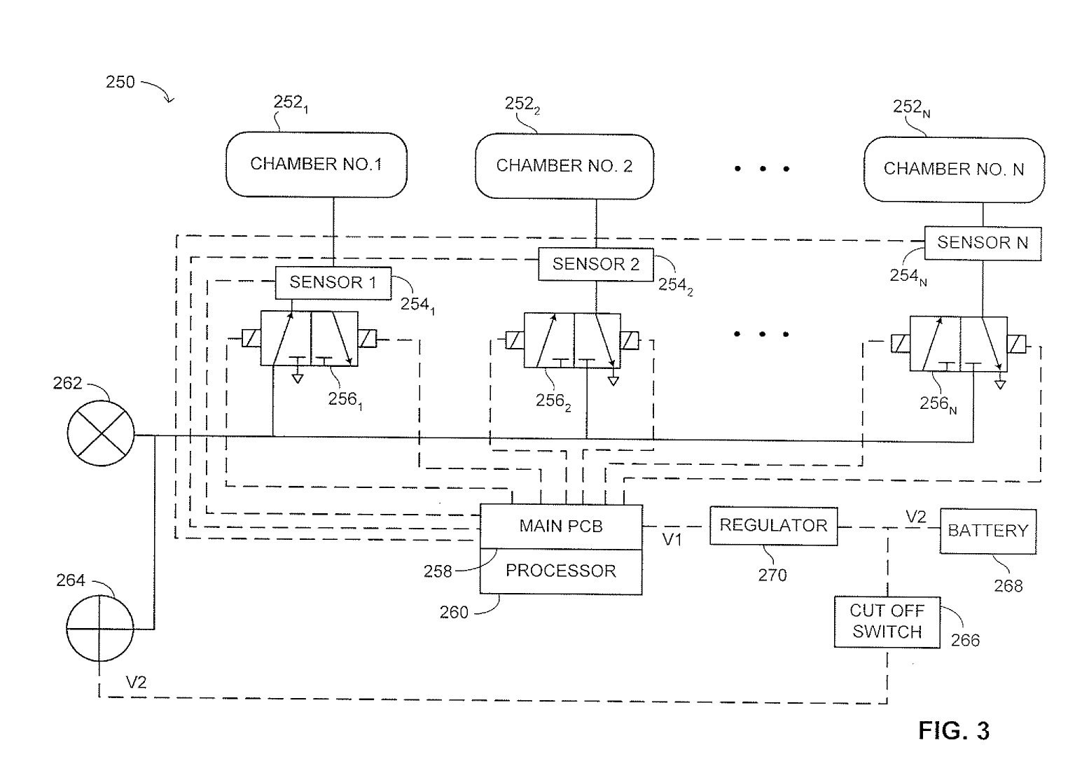

Reference is now made to FIG. 3, which is a schematic illustration of a system, generally referenced 250, constructed and operative in accordance with a further embodiment of the disclosed technique. System 250 is a system for alternately applying and relieving pressure of predetermined portions of a user's foot (not shown), similarly to system 100 of FIGS. 1A and 1B, and system 200 of FIGS. 2A and 2B. System 250 includes a plurality of inflatable chambers 252₁, 252₂ . . . , 252_{N}, a plurality of pressure sensors 254₁, 254₂ . . . , 254_{N}, a plurality of pneumatic valves 256₁, 256₂ . . . , 256_{N}, a main Printed Circuit Board (PCB) 258, a processor 260, a manual pump 262, an electric pump 264, a cut off switch 266, a regulator 270 and a battery 268. Main PCB 258 is coupled with each of pressure sensors 254₁, 254₂ . . . , 254_{N}, each of plurality of pneumatic valves 256₁, 256₂ . . . , 256_{N} (at both command ports thereof, not shown), and with processor 260. The command ports (not shown) of each pneumatic valve are also referred to as solenoids. The plurality of pneumatic valves 256₁, 256₂ . . . , 256_{N} may be considered to form a pneumatic valve assembly (not shown).

Each of pressure sensors 254₁, 254₂ . . . , 254_{N} is coupled between a respective pneumatic valve of valves 256₁, 256₂ . . . , 256_{N}, and a respective chamber of inflatable chambers 252₁, 252₂ . . . , 252_{N}. Battery 268 is coupled with electric pump 264 through cut off switch 266, and with main PCB 258 through regulator 270. Manual pump 262 is coupled with an entry port of each of pneumatic valves 256₁, 256₂ . . . , 256_{N}. Battery 268 provides electric power to main PCB 258, and to electric pump 264. It is noted that each of inflatable chambers 252₁, 252₂ . . . , 252_{N} may include a plurality of chambers, and not a single chamber, as described with reference to FIGS. 1A and 1B.

Each of pneumatic valves 256₁, 256₂ . . . , 256_{N} is an electrically solenoid operated valve, which can change its position from an inflating position to a deflating position according to the electric power supplied to its solenoids. In FIG. 3, continuous lines depict a connection of air flow (e.g., between pump 262 and pneumatic valves 256₁, 256₂ . . . , 256_{N}, and between pneumatic valves 256₁, 256₂ . . . , 256_{N} and inflatable chambers 252₁, 252₂ . . . , 252_{N}). Dashed lines depict an electric connection (e.g., between main PCB 258 and the solenoids of pneumatic valves 256₁, 256₂ . . . , 256_{N}).

Manual pump 262 may be further coupled with a spring (not shown), similarly to spring 108 of FIGS. 1A and 1B, in order to supply air into pump 262. At each given moment, any one of pneumatic valves 256₁, 256₂ . . . , 256_{N} may be in an inflating position, i.e., a position in which air is forwarded into the respective inflatable chamber of chambers 252₁, 252₂ . . . , 252_{N}. Alternatively, none of the valves are in an inflating position, and they are all allowed to deflate. If any of the valves is in an inflating position, pump 262 pumps air into the pneumatic valves, which are in an inflating position, thereby inflating the respective chambers. In the example shown in FIG. 3, valve 256₁ is shown in an inflating position, while the other valves are shown in a deflating position. Each of pressure sensors 254₁, 254₂ . . . , 254_{N} senses the air pressure in the respective one of chambers 252₁, 252₂ . . . , 252_{N}, and provides the respective pressure value to processor 260, via main PCB 258. According to the respective pressure value, processor 260 determines which of pneumatic valves 256₁, 256₂ . . . , 256_{N} are to be in an inflating position, and which in a deflating position (according to a predetermined pressure scheme or cycle). Processor 260 controls the automatic movement of each of pneumatic valves 256₁, 256₂ . . . , 256_{N} into the desired position (i.e., inflating or deflating), by instructing main PCB 258 to provide the necessary electric power to activate the solenoids of pneumatic valves 256₁, 256₂ . . . , 256_{N}. In addition, processor 260 is responsible for determining the cycle characteristics of an alternating pressure scheme, as well as duty cycle and timing of the alternating pressure scheme.

Electric pump 264 is also coupled with an entry port of each of pneumatic valves 256₁, 256₂ . . . , 256_{N}. When the user of system 250 is not walking, manual pump 262 is not operative. In such a case, electric pump 264 pumps air into the pneumatic valves, which are in an inflating position, instead of manual pump 262. For example, when the user is sitting down or resting. During this “rest time” of the user, battery 268 provides electric power to operate electric pump 264, as well as to processor 260.

It is noted that processor 260 may require a certain input voltage V1 (e.g., 6V DC), which is different than the required input voltage V2 of electric pump 264 (e.g., 12V DC). System 250 may include two separate batteries (not shown), each providing electric power for each of processor 260 and electric pump 264. Alternatively, system 250 includes regulator 270, which regulates the voltage V2 from battery 268 to voltage V1 required by processor 260 (e.g., from 12V to 6V).

When the capacity of battery 268 is below a predetermined threshold, cut off switch 266 cuts the power supply to electric pump 264. This is performed in order to save power and to maintain a threshold power to operate processor 260 and maintain system control, although disabling the operation of electric pump 264 during the “rest time”. When the user would start walking again and operate manual pump 262, processor 260 would still be provided with enough power to determine the inflation-deflation cycle of inflatable chambers 252₁, 252₂ . . . , 252_{N}.

Further alternatively, if the battery supplies voltage V1, as required by the processor, the system may further include a charge pump (not shown), coupled between the battery and the cut-off switch. Such charge pump would provide the voltage V2 as required by the electric pump, through the cut-off switch.

According to another embodiment of the disclosed technique, the system may also include a Graphic User Interface (GUI), to allow the user to select a pressure scheme and pneumatic cycle for the inflatable chambers, according to his needs.

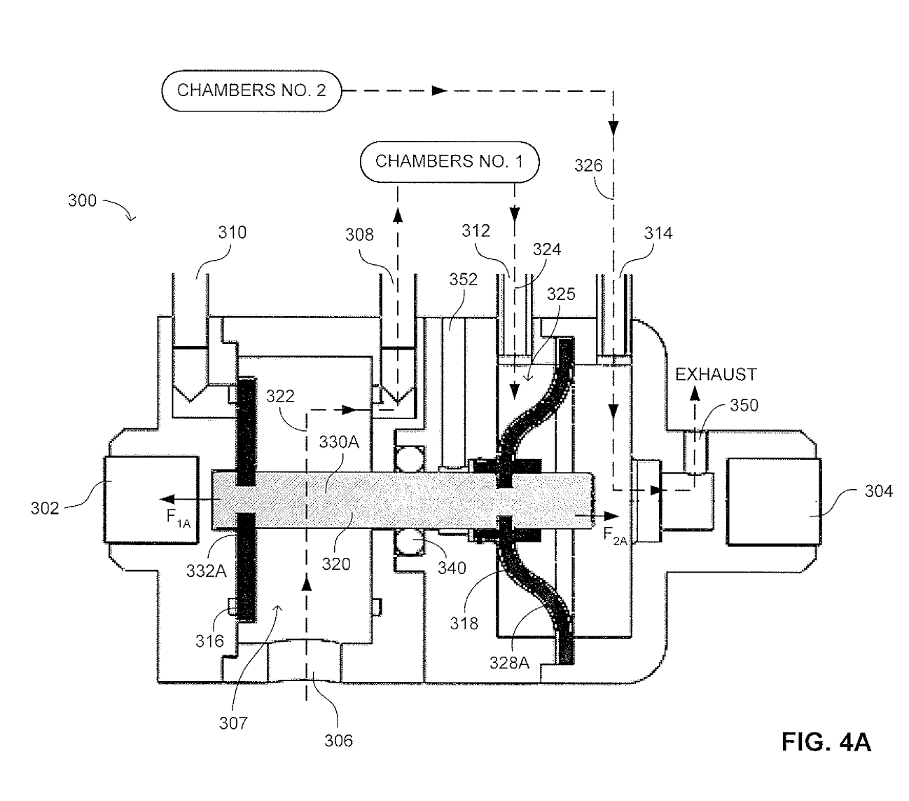

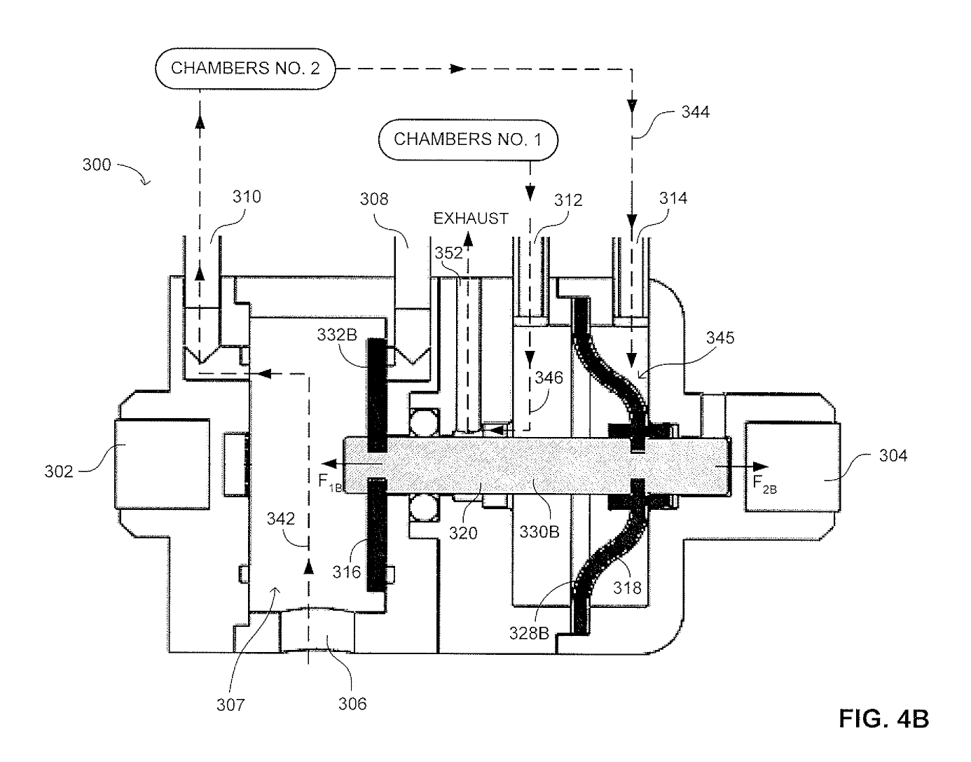

Reference is now made to FIGS. 4A and 4B. FIG. 4A is a schematic illustration side view of a pneumatic control device, generally referenced 300, in a first position, constructed and operative in accordance with another embodiment of the disclosed technique. FIG. 4B is a schematic illustration of the pneumatic control device of FIG. 4A, in a second position. Device 300 may be employed in a system for alternately applying and relieving pressure of predetermined portions of a user's foot (not shown), similarly to system 100 of FIGS. 1A and 1B. Device 300 includes a first magnet 302, a second magnet 304, a device inlet port 306, an inlet cavity 307, a first and a second device outlet ports 308 and 310, a first and a second exhaust inlet ports 312 and 314, a first and a second exhaust outlet ports 350 and 352, a rod 320, a first membrane 316, a second membrane 318 and an O-ring 340.

Device inlet port 306 is coupled with a pump (not shown) for pumping air into device 300 (such as port 114D of FIG. 1A, which is coupled with pump 140 of FIG. 1A). First device outlet port 308 is coupled with an inlet port (not shown) of a first group of inflatable chambers (not shown, such as port 114B of FIG. 1A, coupled with group #1 of chambers 104). Second chamber outlet port 310 is coupled with an inlet port (not shown) of second group of inflatable chambers (not shown, such as port 114A of FIG. 1A, coupled with group #2 of chambers 106). First exhaust inlet port 312 is coupled with an outlet port (not shown) of the first group of inflatable chambers. Second exhaust inlet port 314 is coupled with an outlet port (not shown) of the second group of inflatable chambers. Device inlet port 306 is coupled with inlet cavity 307, which is further coupled with chamber outlet ports 308 and 310. Rod 320 is an elongated rod having a first end and a second end (both not shown). Rod 320 is coupled with first membrane 316 proximate to the first end thereof, and with second membrane 318 proximate to the second end thereof. Rod 320 may be formed of a metallic material, enabling coupling of rod 320 to first magnet 302 at the first end thereof, and by second magnet 304 at the second thereof. It should be noted that magnets are brought herein as mere example, and may be replaced with other known means and methods for coupling of the end of the rods with the device at predetermined positions.

First membrane 316 is moveable between a first position 332A (FIG. 4A) and a second position 332B (FIG. 4B). Second membrane 318 is moveable between a first position 328A (FIG. 4A) and a second position 328B (FIG. 4B). Rod 320 is moveable between a first position 330A (FIG. 4A) and a second position 330B (FIG. 4B).

With reference to FIG. 4A, rod 320 is located at first position 330A, such that the first end thereof is coupled with first magnet 302 (i.e., attracted by the magnet). First membrane 316 is located at first position 332A, such that it prevents passage of air into second chamber outlet 310. In this position, first device outlet 308 is open to inlet cavity 307, such that air may flow from inlet cavity 307 into first device outlet 308. Second membrane 318 is located at first position 328A, such that it forms a cavity 325 for receiving air from first exhaust inlet 312. In this position, second exhaust inlet 314 is open to exhaust port 350, such that air may flow out of the second inflatable chambers (now shown) through second exhaust inlet 314 and further through exhaust outlet port 350, out of device 300. This flow of air is indicated by a dashed arrowed line 326.

In the first position, pumped air flows through inlet 306 into inlet cavity 307. As indicated by a dashed arrowed line 322, the air flows through device outlet 308 into the first group of inflatable chambers (i.e., chamber #1), thereby inflating these chambers. At the same time, air flows from the first group of inflatable chambers, through exhaust inlet 312, into a cavity 325. This flow of air is indicated by a dashed arrowed line 324. The air accumulated in cavity 325 applies pressure on second membrane 318. As a result, second membrane 318 applies force on rod 320, in the direction indicated by force arrow F_{2A}. The magnitude of force F_{2A} increases as more air enters into cavity 325. The direction of force F_{2A} is opposite to the direction of force F_{1A}.

In this position, two forces apply on rod 320: force F_{1A}, originating from the attraction of magnet 302 of rod 320, and force F_{2A}, originating from the air pressure on second membrane 318. As long as the magnitude of force F_{1A} is greater than the magnitude of force F_{2A} (i.e., F_{1A}>F_{2A}), rod 320 remains at first position 330A. Once the magnitude of force F_{2A} is greater than the magnitude of force F_{1A} (i.e., F_{1A}<F_{2A}), rod 320 moves to second position 330B, as depicted in FIG. 4B. When moving between first position 330A and second position 330B, rod 320 rolls along O-ring 340, which surrounds rod 320. When rod 320 moves to second position 330B, it forces the geometry change of first membrane 316 (i.e., flipping the membrane to the second position), which moves to second position 332B, while second membrane moves to second position 328B.

With reference to FIG. 4B, rod 320 is located at second position 330B, such that the second end thereof is coupled with second magnet 304 (i.e., attracted by the magnet). First membrane 316 is located at second position 332B, such that it prevents passage of air into first device outlet 308. In this position, second device outlet 310 is open to inlet cavity 307, such that air may flow from inlet cavity 307 into second device outlet 310. Second membrane 318 is located at second position 328B, such that it forms a cavity 345 for receiving air from exhaust inlet 314. In this position, first exhaust inlet 312 is open to exhaust outlet port 352, such that air may flow out of the first inflatable chambers (now shown) through first exhaust inlet 312 and further through exhaust outlet port 352, out of device 300. This flow of air is indicated by a dashed arrowed line 346.

In the second position, pumped air flows through inlet 306 into inlet cavity 307. As indicated by a dashed arrowed line 342, the air flows through device outlet 310 into the second group of inflatable chambers (i.e., chamber #2), thereby inflating these chambers. At the same time, air flows from the second group of inflatable chambers, through exhaust inlet 314, into a cavity 345. This flow of air is indicated by a dashed arrowed line 344. The air accumulated in cavity 345 applies pressure on second membrane 318. As a result, second membrane 318 applies force on rod 320, in the direction indicated by force arrow F_{1B}. The magnitude of force F_{1B} increases as more air enters into cavity 345. The direction of force F_{1B} is opposite to the direction of force F_{2B}.

In the second position, two forces apply on rod 320: a force F_{2B}, originating from the attraction of magnet 304 of rod 320, and force F_{1B}, originating from the air pressure on second membrane 318. As long as the magnitude of force F_{2B} is greater than the magnitude of force F_{1B} (i.e., F_{2B}>F_{1B}), rod 320 remains at second position 330B. Once the magnitude of force F_{1B} is greater than the magnitude of force F_{1B} (i.e., F_{2B}<F_{1B}), rod 320 moves to first position 330A, as depicted in FIG. 4A. When rod 320 moves to first position 330A, first membrane 316 moves to first position 332A, and second membrane moves to first position 328A. In this manner, device 300 acts as a “flip-flop” mechanism for alternately inflating and deflating the first and second groups of inflatable chambers, by moving between the first and second positions, as elaborated herein above.

It is noted that inlet 306 may be coupled with a manual pump further coupled with an elastic flexible element (e.g., a spring or other specific self retained geometry), located below the heel portion of the foot of a user. The walking action of the user operates on the flexible element to activate the pump (e.g., pressing the flexible element), thereby providing a self-powered compression system. Alternatively, inlet 306 may be coupled with an actuator (not shown) which is further coupled with an electric pump (not shown). The actuator is located below the heel portion of the foot of a user, such that the walking action of the user actuates the actuator to activate the electric pump.

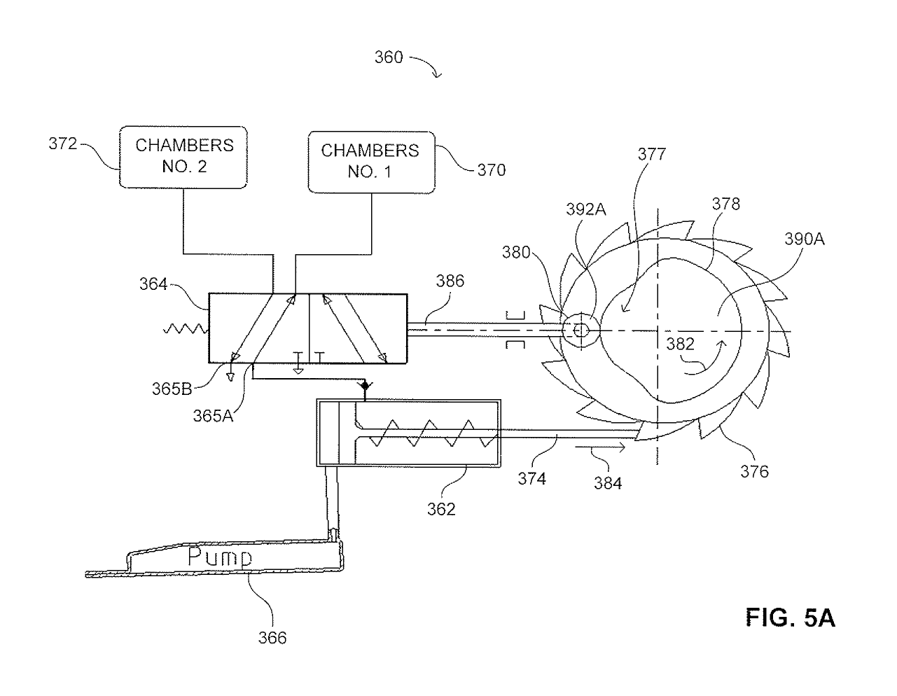

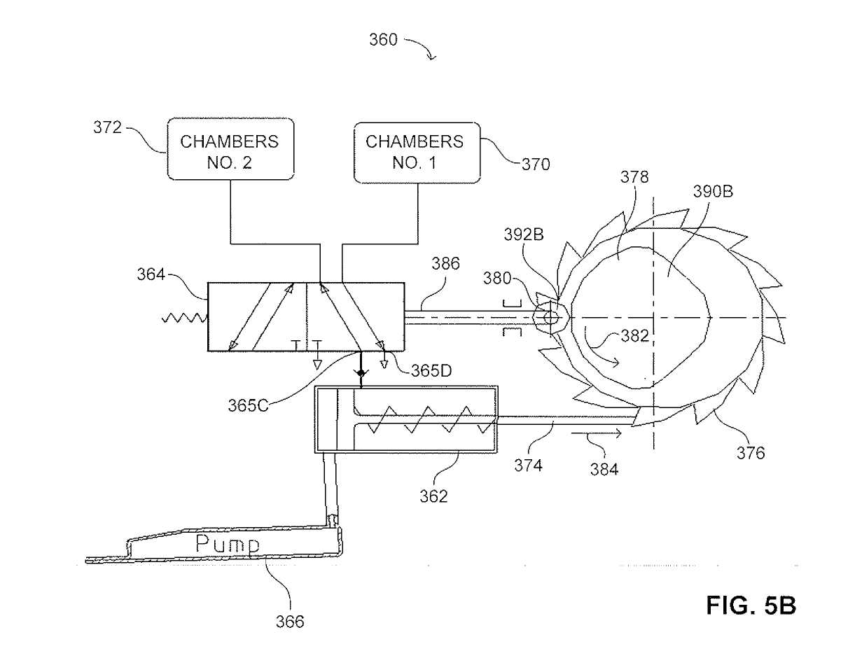

According to another embodiment of the disclosed technique, the system alternately changes the chambers being inflated and deflated after a predetermined number of treads taken by the user. Reference is now made to FIGS. 5A and 5B. FIG. 5A is a schematic illustration of a system, generally referenced 360, in a first position, constructed and operative in accordance with a further embodiment of the disclosed technique. FIG. 5B is a schematic illustration of the system of FIG. 5A, in a second position. System 360 is a system for alternately applying and relieving pressure of predetermined portions of a user's foot (not shown). System 360 includes a first group 370 of inflatable chambers (marked “Chambers No. 1”), a second group 372 of inflatable chambers (marked “Chambers No. 2”), an air pump 366, a pneumatic valve 364, a spring-operated air piston 362, a rod 374, a toothed wheel 376, a ex-centric wheel 378 a lever 380 and a lever rod 386.

Pneumatic valve 364 is coupled with pump 366, lever rod 386, first group 370 of inflatable chambers, and with second group 372 of inflatable chambers. Air pump 366 is coupled with air piston 362, which is further coupled through rod 374 with the teeth (not shown) of toothed wheel 376. Toothed wheel 376 is further firmly coupled with ex-centric wheel 378. Lever 380 is slidably coupled with ex-centric wheel 378 on a first side thereof (not shown), and with a first end (not shown) of lever-rod 386 on the other side thereof (not shown). Ex-centric wheel 378 includes a protruding portion 377. Lever-rod 386 is coupled, at a second end thereof (not shown), with pneumatic valve 364.

Pneumatic valve 364 is depicted as a 5/2 pneumatic valve, wherein two positions are available, a first position (shown in FIG. 5A), and a second position (shown in FIG. 5B). Pump 366 is located under the heel portion of the user's foot, such that when the heel applies pressure on pump 366 (i.e., when the user treads, walks or runs), pump 366 compresses air into piston 362. System 360 is substantially similar to system 100 of FIGS. 1A-1C, in that the inflatable chambers are located below predetermined portions of the user's foot.

Toothed wheel 376, ex-centric wheel 378, lever 380 and lever rod 386, form a ratchet mechanism (not shown), providing continuous movement in the direction of an arrow 382. With reference to FIG. 5A, system 360 is depicted in a first position. Pump 366 compresses air into piston 362, causing rod 374 to move in the direction indicated by an arrow 384. Rod 374 pushes the teeth of toothed wheel 376, thereby turning toothed wheel 376 and ex-centric wheel 378 in the direction indicated by arrow 382. In the first position, ex-centric wheel 378 is located in a first position 390A and lever 380 is located in a first position 392A. Piston 362 transfers the pumped air into an entry port 365A of valve 364. Valve 364 then transfers the air into first group 370 of inflatable chambers, thereby inflating these chambers. At the same time, the chambers of second group 372 of inflatable chambers are deflated to reach ambient pressure, through exhaust port 365B of valve 364. With every tread of the user, rod 374 turns toothed wheel 376 by pressing against another tooth thereof. This is repeated cyclically, until lever 380 passes the protruding portion of ex-centric wheel 378 and moves to the second position thereof (FIG. 5B).

With reference to FIG. 5B, system 360 is depicted in a second position. Ex-centric wheel 378 turns until reaching a second position 390B, such that lever 380 passes the protruding portion of ex-centric wheel 378 and moves to a second position 392B. When lever 380 is in second position 392B, lever-rod 386 pushes against valve 364, such that lever 364 moves to a second position thereof. Pump 366 compresses air into piston 362, which transfers the pumped air into an entry port 365C of valve 364. Valve 364 then transfers the air into second group 372 of inflatable chambers, thereby inflating these chambers. At the same time, the chambers of first group 370 of inflatable chambers are deflated to reach ambient pressure, through exhaust port 365D of valve 364.

Rod 374 still moves in the direction indicated by an arrow 384, pushing against the teeth of toothed wheel 376, thereby turning toothed wheel 376 and ex-centric wheel 378 in the direction indicated by arrow 382. It is noted, that in order for rod 374 to follow the shape of the teeth of toothed wheel 376, rod 374 may be pivotally coupled with piston 362. Alternatively, rod 374 may be formed of an elastic flexible material (e.g., rubber), thereby allowing rod 374 to bend while following the shape of the teeth of toothed wheel 376. With every tread of the user, rod 374 turns toothed wheel 376 by pressing against another tooth thereof. This is repeated cyclically, until lever 380 reaches the protruding portion of ex-centric wheel 378 and moves back to the first position thereof (FIG. 5A).

In this manner, the inflating and deflating of groups of inflatable chambers changes every predetermined number of treads taken by the user. The number of treads is determined according to the size of the toothed wheel, the size of the teeth thereof, and the size of the ex-centric wheel.

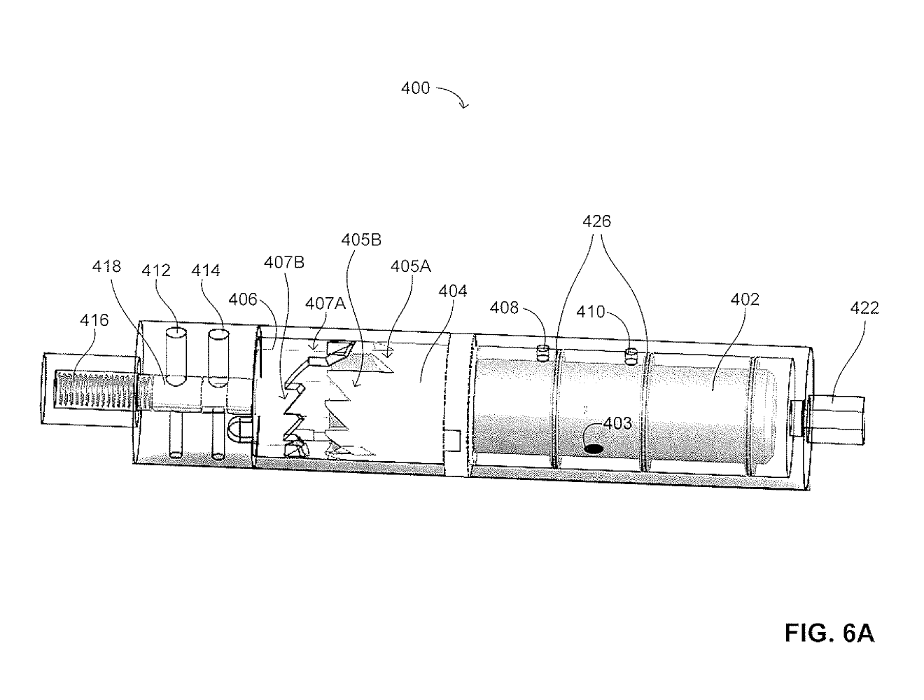

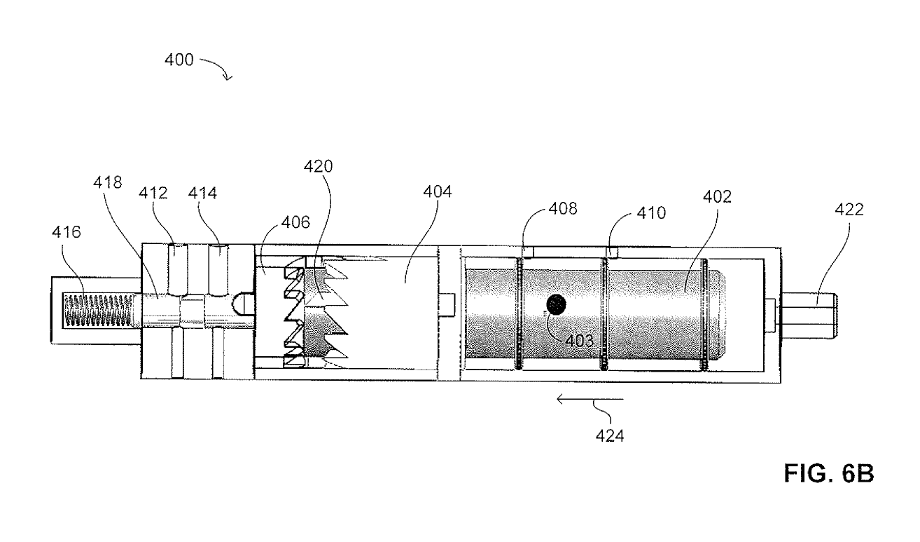

Reference is now made to FIGS. 6A and 6B. FIG. 6A is a perspective view schematic illustration of a device, generally referenced 400, in a first position, constructed and operative in accordance with another embodiment of the disclosed technique. FIG. 6B is a side view schematic illustration of the device of FIG. 6A, in a second position. Device 400 may be employed in a system for alternately applying and relieving pressure of predetermined portions of a user's foot (not shown), similarly to system 100 of FIGS. 1A and 1B. Device 400 includes an air piston 402, a first toothed cylinder 404, a second toothed cylinder 406, a piston air outlet 403, a first chamber inflating outlet 408, a second chamber inflating outlet 410, a first chamber deflating outlet 414, a second chamber deflating outlet 412, a rod 418, a spring 316 and an air inlet 422.

First toothed cylinder 404 further includes a first level 405A of triangular teeth and a second level 405B of triangular teeth. First level 405A and second level 405B are separated by a predetermined distance. Second toothed cylinder 406 further includes a first level 407A of triangular teeth and a second level 407B of triangular teeth. First level 407A and second level 407B are separated by a predetermined distance, equal to the distance between the levels of first toothed cylinder 404. Piston 402 further includes a piston air outlet (not shown) and a guiding tooth 420 (shown in FIG. 6B), which interacts either with first toothed cylinder 404 or with second toothed cylinder 406. The direction of the teeth of toothed cylinder 404 is opposite the direction of guiding tooth 420, in order to force rotating by predetermined increments on guiding tooth 420 and thereby rotating piston 402 and rod 418. Piston 402 is surrounded by a plurality of sealing membranes 426 (e.g., O-rings surrounding piston 402), separated from one another by a distance substantially equal to the distance between first and second chamber inflating outlets 408 and 410. Each of sealing membranes 426 is located on another side of piston air outlet 403.

Air inlet 422 is coupled with an air pump (not shown), similar to air pump 366 of FIG. 5A. Piston 402 is firmly coupled with rod 418, which is coupled with an end of spring 416. First chamber inflating outlet 408 is coupled with an inlet port of a first group of inflatable chambers (not shown) and second chamber inflating outlet 410 is coupled with an inlet port of a second group of inflatable chambers (not shown). First chamber deflating outlet 414 is coupled with an outlet port of the first group of inflatable chambers, and second chamber deflating outlet 412 is coupled with an outlet port of the second group of inflatable chambers. Inflating outlets 408 and 410 provide air arriving from the pump to the respective inflatable chambers. Deflating outlets 412 and 414 allow air to deflate from one of the groups of inflatable chambers, when the other group is being inflated, through the piston air outlet. First toothed cylinder 404 and second toothed cylinder 406, form a ratchet mechanism (not shown).

When the user presses on the pump (e.g., while treading), incoming air pushes piston 402 in the direction of arrow 424 (FIG. 6B). Piston 402 then causes guiding tooth 420 to interact with the teeth of second toothed cylinder 406. Meanwhile, piston air outlet 403 allows incoming air to exit piston 402 toward either one of chamber inflating outlets 408 and 410. At the same time, one of deflating outlets 412 and 414 allow air to exit from one of the groups of inflatable chambers, when the other group is being inflated. The location of sealing membranes 426 relative to piston air outlet 403 allows air to flow only to one inflating outlet in each position, and preventing air from flowing into the other inflating outlet.

In the first position shown in FIG. 6A, guiding tooth 420 interacts with first level 407A of second toothed cylinder 406. Thus, piston 402 is located proximate to inlet 422, such that inflating outlet 410 is located between sealing membranes 426, allowing inflating of the respective inflatable chambers (and therefore deflating of the chambers respective of outlet 408). The interaction between guiding tooth 420 and first level 407A of second toothed cylinder 406 causes rotation of piston 402 by a predetermined angle. Spring 416 applies sufficient pressure on piston 402 (through rod 418) to maintain piston 402 in the same position relative to inflating outlets 408 and 410, after each rotation (i.e., after passing another tooth of second toothed cylinder 406). Device 400 remains in the first position until guiding tooth 420 passes to interact with second level 427B of second toothed cylinder 406. It is noted, that with each tread of the user, piston 402 causes guiding tooth 420 to pass another tooth of second toothed cylinder 406. Therefore, device 400 will change from the first position to the second position (and vice versa), according to the number of teeth on each of the levels of second toothed cylinder 406.

FIG. 6B depicts the second position of device 400. In this position, guiding tooth 420 interacts with second level 407B of second toothed cylinder 406. Thus, piston 402 is located farther from inlet 422, such that inflating outlet 408 is located between sealing membranes 426, allowing inflating of the respective inflatable chambers (and therefore deflating of the chambers respective of outlet 410). The interaction between guiding tooth 420 and second level 407B of second toothed cylinder 406 causes rotation of piston 402 by a predetermined angle. Spring 416 applies sufficient pressure on piston 402 (through rod 418) to maintain piston 402 in the same position relative to inflating outlets 408 and 410, after each rotation (i.e., after passing another tooth of second toothed cylinder 406). Device 400 remains in the second position until guiding tooth 420 passes to interact with first level 427A of second toothed cylinder 406.

In this manner, the inflating and deflating of groups of inflatable chambers changes every predetermined number of treads taken by the user. The number of treads is determined according to the number of teeth and geometry of the first and second cylinders.

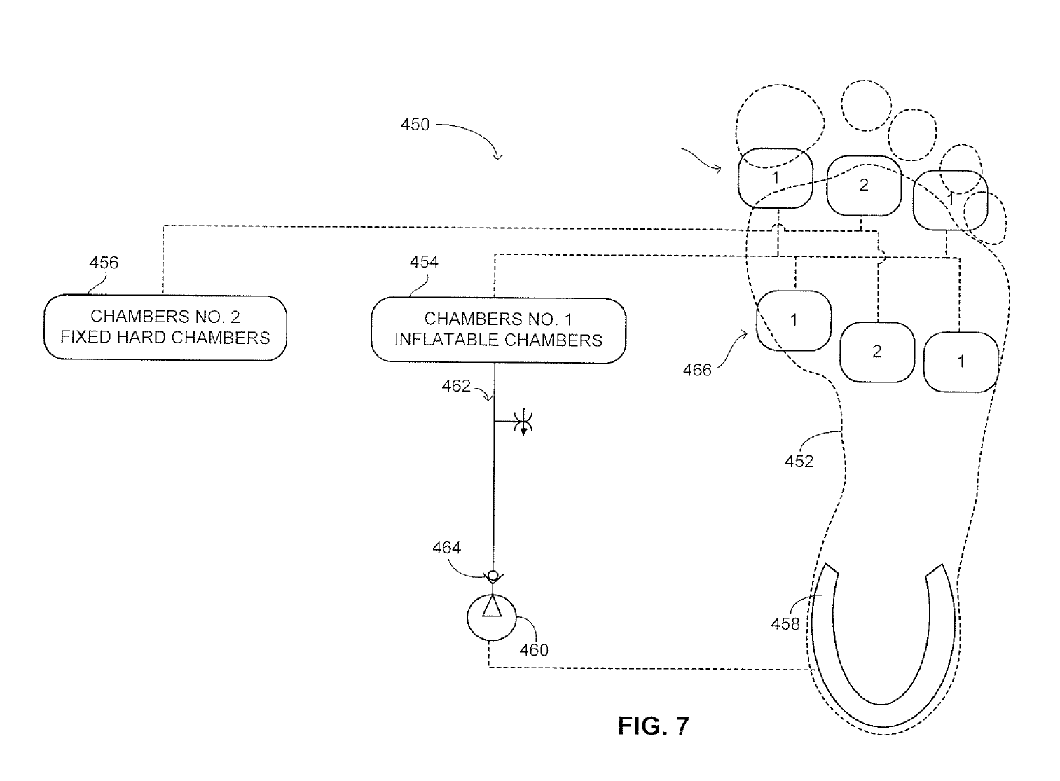

Reference is now made to FIG. 7, which is a schematic illustration of a system, generally referenced 450, constructed and operative in accordance with a further embodiment of the disclosed technique. System 450 is a system for alternately applying and relieving pressure of predetermined portions of a user's foot 452, similar to system 100 of FIG. 1A. System 450 includes a group 454 of inflatable chambers (marked “No. 1”), a group 456 of fixed hard (i.e., non-inflatable) chambers (marked “No. 2”), a spring 458, an air pump 460, a regulator 462, a check valve 464, and a housing (not shown). Spring 458 is coupled with pump 460. Manual pump 460 is coupled with spring 458, and with group 454 of inflatable chambers, through check valve 464 and regulator 462. The components of system 450, except for the inflatable chambers, are located within the housing. The housing may be located, for example, within a sole of a shoe, such as shown in FIG. 1C, worn by the user.

Spring 458 is located below a heel region (not shown) of foot 452 of a user. Spring 458 may be a folding spring, similar to spring 108 of FIG. 1B, moving according to pressure applied thereon by the heel of the foot. Alternatively, spring 458 may be replaced with any other known elastic flexible element, which would operate pump 460, when pressure is applied thereon. The chambers of group 454 of inflatable chambers and of group 456 of fixed hard chambers are located below a front region 468 and a center region 466 of foot 452. The chambers of group 454 of inflatable chambers may be inflated with air driven by manual pump 460. The chambers of group 456 of fixed hard chambers are fixed non-inflatable chambers, which keep their shape under pressure (i.e., lumps). Alternatively, the chambers of group 456 of fixed hard chambers may have a certain degree of flexibility, when pressure is applied thereon.

When the heel of foot 452 applies pressure on spring 458 (i.e., when the user treads, walks or runs), spring 458 compresses air into pump 460. Pump 460 pumps the compressed air and forwards the air into chambers of group 454 of inflatable chambers, through check valve 464, thereby inflating these chambers. Check valve 464 prevents air from flowing back toward pump 460. The compressed air is also supplied to regulator 462. When no pressure is applied on spring 458, for example when the user does not walk (i.e., rests or stands), the chambers of group 454 of inflatable chambers are not inflated. Instead, these chambers are deflated slowly through regulator 462.

In this manner, the groups of chambers are alternately inflated and deflated, according to whether pressure is applied by the heel of the foot or not. Whenever the inflatable chambers are inflated or deflated, pressure is applied to- and relieved off- of the corresponding parts of the user's foot. For example, such alternate pressure application and relief is useful for treating or relieving pain induces by diabetic foot ulcers of the user. Alternatively, such alternate pressure application and relief may be employed by a healthy user to promote blood flow in the foot or simply providing an enjoyable feeling to the user.

It is noted, that according to an alternative embodiment, system 450 may include only inflatable chambers, such as the chambers of group 454 of inflatable chambers. Further alternatively, system 450 may include only fixed hard chambers, such as the chambers of group 456 of fixed hard chambers.

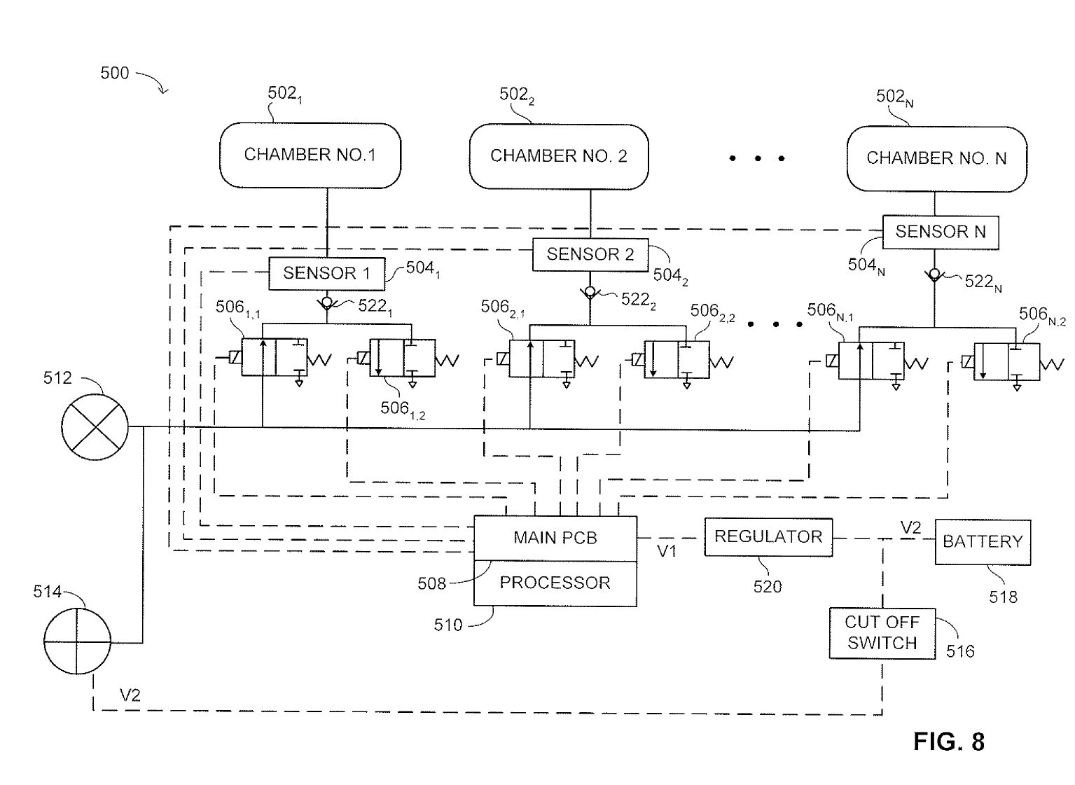

Reference is now made to FIG. 8, which is a schematic illustration of a system, generally referenced 500, constructed and operative in accordance with another embodiment of the disclosed technique. System 500 is a system for alternately applying and relieving pressure of predetermined portions of a user's foot (not shown), similarly to system 100 of FIGS. 1A and 1B, and system 200 of FIGS. 2A and 2B. System 500 includes a plurality of inflatable chambers 502₁, 502₂ . . . , 502_{N}, a plurality of pressure sensors 504₁, 504₂ . . . , 504_{N}, a plurality of pneumatic valves 506_{1,1}, 506_{1,2} . . . , 506_{N,1}, 506_{N,2}, a plurality of check valves 522₁, 522₂ . . . , 522_{N}, a main Printed Circuit Board (PCB) 508, a processor 510, a manual pump 512, an electric pump 514, a cut off switch 516, a regulator 520 and a battery 518. Main PCB 518 is coupled with each of pressure sensors 504₁, 504₂ . . . , 504_{N}, each of plurality of pneumatic valves 506_{1,1}, 506_{1,2}, 506_{2,1}, 506_{2,2} . . . , 506_{N,1}, 506_{N,2} (each at the command port thereof, not shown), and with processor 510. The command ports (not shown) of each pneumatic valve are also referred to as solenoids. The plurality of pneumatic valves 506_{1,1}, 506_{1,2}, 506_{2,1}, 506_{2,2} . . . , 506_{N,1}, 506_{N,2} may be considered to form a pneumatic valve assembly (not shown).

Each of pressure sensors 504₁, 504₂ . . . , 504_{N} is coupled between a respective pair of pneumatic valves (506_{1,1}, 506_{1,2}), (506_{2,1}, 506_{2,2}) . . . , (506_{N,1}, 506_{N,2}), and a respective chamber of inflatable chambers 502₁, 502₂ . . . , 502_{N}, through a respective one of check valves 522₁, 522₂ . . . 522_{N}. Battery 518 is coupled with electric pump 514 through cut off switch 516, and with main PCB 508 through regulator 520. Manual pump 512 is coupled with an entry port (not shown) of the first of each pair of pneumatic valves, i.e., pneumatic valves 506_{1,1}, 506_{2,1}, . . . , 506_{N,1}. Battery 518 provides electric power to main PCB 508, and to electric pump 514. It is noted that each of inflatable chambers 502₁, 502₂ . . . , 502_{N} may include a plurality of chambers, and not a single chamber, as described with reference to FIGS. 1A and 1B.

Each of pneumatic valves 506_{1,1}, 506_{1,2}, 506_{2,1}, 506_{2,2} . . . , 506_{N,1}, 506_{N,2} is an electrically 2×2 solenoid operated valve. It should be noted that although FIG. 8 depicts these pneumatic valves as spring-returned valves, some of the valves (or all) may alternatively be solenoid-solenoid valves. Each pair of pneumatic valves (506_{1,1}, 506_{1,2}), (506_{2,1}, 506_{2,2}) . . . , (506_{N,1}, 506_{N,2}), can change its position between an inflating position, a deflating position, and a holding position, according to the electric power supplied to the solenoids of the respective valves of that couple. In the inflating position, the respective inflatable chambers are inflated with air. In the deflating position, the respective chambers are deflated. In the holding position, the respective chambers are maintained in an inflated position, preventing air from being deflated or inflated thereof. In FIG. 8, continuous lines depict a connection of air flow (e.g., between pump 512 and pneumatic valves 506_{1,1}, 506_{1,2}, 506_{2,1}, 506_{2,2} . . . , 506_{N,1}, 506_{N,2}, and between pneumatic valves 506_{1,1}, 506_{1,2}, 506_{2,1}, 506_{2,2} . . . , 506_{N,1}, 506_{N,2} and inflatable chambers 502₁, 502₂ . . . , 502_{N}). Dashed lines depict an electric connection (e.g., between main PCB 508 and the solenoids of pneumatic valves 506_{1,1}, 506_{1,2}, 506_{2,1}, 506_{2,2 . . . , 506}_{N,1}, 506_{N,2}).

Manual pump 512 may be further coupled with a spring (not shown), similarly to spring 108 of FIGS. 1A and 1B, in order to supply air into pump 512. At each given moment, any one of pneumatic valves pairs (506_{1,1}, 506_{1,2}), (506_{2,1}, 506_{2,2}) . . . , (506_{N,1}, 506_{N,2}) may be in an inflating position, i.e., a position in which air is forwarded into the respective inflatable chamber of chambers 502₁, 502₂ . . . , 502_{N}. Alternatively, none of the valve pairs are in an inflating position, and all of the chambers are allowed to deflate, in order to enable maximum flexibility. If any of the valves is in an inflating position, pump 512 pumps air into the first one of the pneumatic valve pairs, which are in an inflating position, thereby inflating the respective chambers. In the example shown in FIG. 8, all of the valve pairs are shown in the inflating position. Each of pressure sensors 504₁, 504₂ . . . , 504_{N} senses the air pressure in the respective one of chambers 502₁, 502₂ . . . , 502_{N}, and provides the respective pressure value to processor 510, via main PCB 508. According to the respective pressure value, processor 510 determines which of pneumatic valve pairs are to be in an inflating position, which in a deflating position, and which in a holding position (according to a predetermined pressure scheme or cycle). Processor 510 controls the automatic movement of each of pneumatic valves 506_{1,1}, 506_{1,2}, 506_{2,1}, 506_{2,2} . . . , 506_{N,1}, 506_{N,2} into the desired position (i.e., inflating, deflating or holding), by instructing main PCB 508 to provide the necessary electric power to activate the solenoids of the pneumatic valves. In addition, processor 510 is responsible for determining the cycle characteristics of an alternating pressure scheme, as well as duty cycle and timing of the alternating pressure scheme.

Electric pump 514 is also coupled with an entry port of the first one of each of pneumatic valves pairs (506_{1,1}, 506_{1,2}), (506_{2,1}, 506_{2,2}) . . . , (506_{N,1}, 506_{N,2}). When the user of system 500 is not walking, manual pump 512 is not operative. In such a case, electric pump 514 pumps air into the pneumatic valves, which are in an inflating position, instead of manual pump 512. For example, when the user is sitting down or resting. During this “rest time” of the user, battery 508 provides electric power to operate electric pump 514, as well as to processor 510.

It is noted that processor 510 may require a certain input voltage V1 (e.g., 6V DC), which is different than the required input voltage V2 of electric pump 514 (e.g., 12V DC). System 500 may include two separate batteries (not shown), each providing electric power for each of processor 510 and electric pump 514. Alternatively, system 500 includes regulator 520, which regulates the voltage V2 from battery 518 to voltage V1 required by processor 510 (e.g., from 12V to 6V).

When the capacity of battery 518 is below a predetermined threshold, cut off switch 516 cuts the power supply to electric pump 514. This is performed in order to save power and to maintain a threshold power to operate processor 510 and maintain system control, although disabling the operation of electric pump 514 during the “rest time”. When the user would start walking again and operate manual pump 512, processor 510 would still be provided with enough power to determine the inflation-deflation-holding cycle of the inflatable chambers.

Further alternatively, if the battery supplies voltage V1, as required by the processor, the system may further include a charge pump (not shown), coupled between the battery and the cut-off switch. Such charge pump would provide the voltage V2 as required by the electric pump, through the cut-off switch.

According to another embodiment of the disclosed technique, the system may also include a Graphic User Interface (GUI), to allow the user to select a pressure scheme and pneumatic cycle for the inflatable chambers, according to his needs.

### INCORPORATION BY REFERENCE

The entire disclosure of each of the publications and patent documents referred to herein is incorporated by reference in its entirety for all purposes to the same extent as if each individual publication or patent document were so individually denoted.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A system for alternately applying and relieving pressure of predetermined portions of a foot of a user, the system comprising:
a plurality of inflatable chambers, located at predetermined locations, corresponding to said predetermined portions of said foot;
a pump;
a center pneumatic valve, coupled with said pump and with said inflatable chambers, for receiving air from said pump and inflating at least one of said inflatable chambers; and
a pneumatic valve assembly, coupled with said center pneumatic valve, said pneumatic valve assembly being coupled with at least a respective one of said inflatable chambers, said pneumatic valve assembly being moveable between two positions, said pneumatic valve assembly changing position when a predetermined pressure value is achieved in said corresponding inflatable chamber, when said pneumatic valve assembly changes position said center pneumatic valve changes position, thereby inflating another inflatable chamber.

2. The system of claim 1, wherein said pneumatic valve assembly includes a plurality of pneumatic valves, each coupled with a respective one of said inflatable chambers.

3. The system of claim 1, further comprising a plurality of regulators, each coupled between one of said plurality of pneumatic valves and a respective inflatable chamber, each of said regulators determining the pressure value of the air in said respective inflatable chamber.

4. The system of claim 1, further comprising a check valve coupled between said pump and said center pneumatic valve, for preventing air from flowing back toward said pump.

5. The system of claim 1, further comprising a flexible element, located below the heel region of said foot and coupled with said pump, said flexible element compressing air into said pump when the heel of said foot applies pressure on said flexible element.

6. The system of claim 5, wherein said flexible element is selected from the group consisting of a spring and a self retained geometry element.

7. A system for alternately applying and relieving pressure of predetermined portions of a foot of a user, the system comprising:
a plurality of inflatable chambers, located at predetermined locations, corresponding to said predetermined portions of said foot;
a pneumatic valve assembly, coupled with at least a respective one of said inflatable chambers, said pneumatic valve assembly including at least an entry port and electric command ports for each one of said inflatable chambers, said pneumatic valve assembly being movable between an inflating position and a deflating position for each one of said inflatable chambers;
a plurality of pressure sensors, each coupled with a respective inflatable chamber, for providing a pressure value of the pressure in said respective inflatable chamber;
a pump, coupled with an entry port of each of said pneumatic valve assembly, for providing air into said valve assembly, and inflating at least one of said inflatable chambers; and
a processor, coupled with each of said pressure sensors, and with each of said electric command ports, for determining if said pneumatic valve assembly is to be in an inflating position or in a deflating position for each one of said inflatable chambers, according to the respective pressure value of each of said inflatable chambers, said processor automatically moving said pneumatic valve assembly into the desired position by providing the necessary electric power to said command ports.

8. The system of claim 7, wherein said pneumatic valve assembly includes a plurality of pneumatic valves, each coupled with a respective one of said inflatable chambers.

9. The system of claim 7, further comprising:
an electric pump, coupled with an entry port of said pneumatic valve assembly, for providing air into said valve assembly, and inflating at least one of said inflatable chambers, when said pump is not operative;
a battery for supplying power to said electric pump and to said processor;
a regulator for regulating the power from said battery to said processor; and
a cut off switch, for cutting off the power from said battery to said electric pump, when the capacity of said battery is below a predetermined threshold.

10. A device for use in a system for alternately applying and relieving pressure of predetermined portions of a foot of a user, said device being moveable between a plurality of positions, the system including a plurality of inflatable chambers, located at predetermined locations, corresponding to said predetermined portions of said foot, the device comprising:
a plurality of membranes;
an elongated rod, having a first end and a second end, said rod being coupled with said a first membrane proximate to said first end thereof, said rod being coupled with a second membrane proximate to said second end thereof, said rod being moveable between a plurality of positions;
a plurality of fasteners, each fastener maintaining said rod in another one of said plurality of positions;
a device inlet, coupled with a pump for pumping air into said device inlet;
a plurality of device outlets, each coupled with a respective inflatable chamber, for guiding pumped air into said respective inflatable chamber, when said device outlet is open; and
a plurality of exhaust inlets, each coupled with a respective inflatable chamber, for exhausting air from said respective inflatable chamber out of said device, when said chamber inlet is open,
wherein said device moves between said plurality of positions, according to the air pressure in said inflatable chambers, each of said membranes allowing or preventing air passage to said inflatable chambers, according to the position the device is at.

11. The device of claim 10, wherein said fasteners include magnets, said elongated rod is formed of a metallic material, said rod being coupled with another one of said magnets during another one of said positions.

12. The device of claim 10, wherein said plurality of positions includes two positions.

13. A device for use in a system for alternately applying and relieving pressure of predetermined portions of a foot of a user, said device being moveable between a plurality of positions, the system including a plurality of inflatable chambers, located at predetermined locations, corresponding to said predetermined portions of said foot, the system further including a pneumatic valve, having a plurality of outlets, each coupled with a respective inflatable chamber, for guiding air into a respective inflatable chamber, when the outlet is open, the device comprising:
a ratchet mechanism, for moving said pneumatic valve from one inflatable chamber to another;
an air piston, coupled with a pump for pumping air into said device, said air piston including an air outlet for providing air into an inlet of said pneumatic valve; and
an elongated rod, having a first end and a second end, the first end of said rod being coupled with said air piston, the second end of said rod interacting with said ratchet mechanism, thereby rotating said ratchet mechanism by predetermined increments, when sufficient pressure is applied on said rod by said air piston;
wherein said device moves between said positions, according to said ratchet mechanism.

14. The device of claim 13, wherein said ratchet mechanism includes:
a toothed wheel, having a plurality of teeth on the outer circumference thereof;
an excentric wheel, including a protruding portion, said excentric wheel being firmly coupled with said toothed wheel, for rotating along with said toothed wheel; and
a lever for interacting with said excentric wheel, said lever being coupled with a lever-rod at a first end thereof, said lever-rod being coupled with said pneumatic valve at a second end thereof;
wherein the second end of said elongated rod interacts with said teeth of said toothed wheel by pressing against said teeth, thereby rotating said toothed wheel and said excentric wheel by predetermined increments, when sufficient pressure is applied on said rod by said air piston, and
wherein said device moves between said positions, according to the position of said lever relative to said excentric wheel.

15. The device of claim 13, wherein said ratchet mechanism moves said pneumatic valve from one inflatable chamber to another after a predetermined number of treads of said user.

16. The device of claim 14, wherein said second end of said elongated rod rotates said toothed wheel and said excentric wheel by a predetermined increment, with each tread of said user, said ratchet mechanism moving said pneumatic valve from one inflatable chamber to another after a predetermined number of treads of said user.

17. A device for use in a system for alternately applying and relieving pressure of predetermined portions of a foot of a user, said device being moveable between two positions, the system including a plurality of inflatable chambers, located at predetermined locations, corresponding to said predetermined portions of said foot, the device comprising:
an air piston, coupled with an air inlet of said device, said air piston including a guiding tooth and a piston air outlet, for letting air out of said piston;
an elongated rod, firmly coupled with said air piston;
a ratchet mechanism, slidably coupled with said air piston and said elongated rod, for interacting with said guiding tooth of said piston at each of said positions of said device;
a first chamber inflating outlet, coupled with an inlet of a first plurality of said inflatable chambers, for moving air from said piston toward said first inflatable chambers, when said device is at said first position;
a second chamber inflating outlet, coupled with an inlet of a second plurality of said inflatable chambers, for moving air from said piston toward said second inflatable chambers, when said device is at said second position;
a first chamber deflating outlet, coupled with an outlet of said first plurality of said inflatable chambers, for deflating air from said first inflatable chambers, when said device is at said second position; and
a second chamber deflating outlet, coupled with an outlet of said second plurality of said inflatable chambers, for deflating air from said second inflatable chambers, when said device is at said first position;
wherein said guiding tooth interacts with said ratchet mechanism, thereby rotating said ratchet mechanism by predetermined increments, said device moving between said positions, according to the position of said piston relative to said chamber inflating outlets.

18. The device of claim 17, wherein said ratchet mechanism includes a first toothed cylinder and a second toothed cylinder, each having a plurality of teeth at a first level and at a second level thereof, and
wherein said guiding tooth interacts with said teeth of said toothed cylinders by pressing against said teeth, thereby rotating either one of said toothed cylinders by predetermined increments.

19. The device of claim 17, wherein said ratchet mechanism moves said device between said positions after a predetermined number of treads of said user.

20. The device of claim 16, wherein said guiding tooth interacts with said ratchet mechanism, thereby rotating said ratchet mechanism by a predetermined increment, with each tread of said user, said ratchet mechanism moving said device between said positions after a predetermined number of treads of said user.

21. A system for applying and relieving pressure of predetermined portions of a foot of a user, the system comprising:
a pump;
a check valve, coupled with said pump;
a regulator; and
at least one inflatable chamber, located at a predetermined location, corresponding to one of said predetermined portions of said foot, said at least one inflatable chamber being coupled with said pump, said check valve and with said regulator, said pump inflating air into said inflatable chamber when the user is treading on said foot, said inflatable chamber being deflated through said regulator when the user is not treading on said foot.

22. The system of claim 21, further comprising at least one fixed chamber, located at a predetermined location, corresponding to one of said predetermined portions of said foot, said fixed chamber applying pressure on said respective predetermined portion, when said foot is treading.

23. A system for alternately applying and relieving pressure of predetermined portions of a foot of a user, the system comprising:
a plurality of inflatable chambers, located at predetermined locations, corresponding to said predetermined portions of said foot;
a pneumatic valve assembly, coupled with at least a respective one of said inflatable chambers, said pneumatic valve assembly including at least an entry port and electric command ports for each one of said inflatable chambers, said pneumatic valve assembly being movable between an inflating position, a deflating position and holding position, for each one of said inflatable chambers;
a plurality of pressure sensors, each coupled with a respective inflatable chamber, for providing a pressure value of the pressure in said respective inflatable chamber;
plurality of check valves, each coupled between said pneumatic valve assembly and a respective inflatable chamber;
a pump, coupled with an entry port of each of said pneumatic valve assembly, for providing air into said valve assembly, and inflating at least one of said inflatable chambers; and
a processor, coupled with each of said pressure sensors, and with each of said electric command ports, for determining if said pneumatic valve assembly is to be in an inflating position, in a deflating position, or in a holding position, for each one of said inflatable chambers, according to the respective pressure value of each of said inflatable chambers, said processor automatically moving said pneumatic valve assembly into the desired position by providing the necessary electric power to said command ports.

24. The system of claim 23, wherein said pneumatic valve assembly includes a plurality of pneumatic valve pairs, each pair being coupled with a respective one of said inflatable chambers, each pneumatic valve pair including a first pneumatic valve and a second pneumatic valve, wherein the entry port of said first pneumatic valve is coupled with said pump, the command port of said first pneumatic valve and the command port of second pneumatic valve are coupled with said processor.
